(19) United States
(12) Patent Application Publication
(10) Pub. No.: US 2026/0232347 A1
(43) Pub. Date: Aug. 13, 2026

(54) CANNULA ASSEMBLY FOR PROVIDING ENHANCED NAVIGATION IN A SURGICAL SITE

(71) Applicant: NEW VIEW SURGICAL, Newton Center, MA (US)

(72) Inventors: Bryce C. Klontz, JR., Boston, MA (US); Joseph Peter Corrigan, Hardwick, Cambridgeshire (GB); Joshua John Gibson, Needingworth, Cambridge (GB); Rachel Mary Rakvica, Newbury, MA (US)

(73) Assignee: New View Medical, Inc., Newton Center, MA (US)

(21) Appl. No.: 19/157,432

(22) PCT Filed: Mar. 7, 2024
(86) PCT No.: PCT/US2024/018790

§ 371 Date: Aug. 18, 2025

Related U.S. Application Data
(60) Provisional application No. 63/489,487, filed on Mar. 10, 2023.

(51) Int. Cl. A61B 17/34; A61B 1/313; A61B 34/10; A61B 34/20

(52) U.S. Cl. CPC A61B 17/3423; A61B 1/3132; A61B 34/20; A61B 2034/107

(57) ABSTRACT

A cannula assembly is disclosed for performing a surgical procedure on a patient. The cannula assembly may include a cannula tube having a longitudinal axis, a proximal end portion and a distal end portion configured for insertion into a patient. The cannula tube may have a housing coupled thereto so as to be positioned within the patient when the distal end of the cannula tube is inserted into the patient. The housing may be movable relative to the cannula tube between a closed position and an open position. The housing may include a light source and an image sensor configured to provide a first image data stream of the patient's anatomy and a surgical tool when the housing is in the open position within the patient. A processor may be configured to the process the first data image stream and to generate navigation data based on the image data stream.

### CROSS-REFERENCE TO RELATED APPLICATIONS

This application claims the benefit of U.S. Provisional Application Ser. No. 63/489,487, filed Mar. 10, 2023, the complete disclosure of which is incorporated herein by reference for all purposes.

### BACKGROUND

Minimally invasive surgery involves making small incisions into a body of a patient to insert surgical tools. For example, a surgeon may perform a laparoscopic procedure using multiple cannulas inserted through individual incisions that accommodate various surgical tools, including illumination devices and imaging devices. To accomplish the insertion, cannula assemblies may be used to puncture the body cavity. A cannula assembly often includes an obturator and a cannula. An obturator is a device placed inside a cannula, the obturator having either a sharp tip (e.g., a pointed cutting blade) or a blunt tip for creating an incision or opening in the patient for the cannula to pass through. After the obturator and cannula are inserted, the obturator is removed, leaving the cannula in place for use in inserting the surgical tools into the surgical space within a patient. Typically, in addition to cannulas forming individual incisions for surgical tools, an individual incision may also be made through the patient by a cannula that is thereafter dedicated to holding an illumination and/or imaging device, e.g., a traditional endoscope or laparoscope. A surgical tool combining a cannula and an imaging device in a single unit is disclosed, for example, in U.S. Pat. No. 8,834,358, the disclosure of which is herein incorporated by reference in its entirety.

### SUMMARY

In accordance with various embodiments, there is provided a surgical system for performing a surgical procedure on a patient using a surgical tool. The surgical system may include a cannula assembly including a cannula tube having a longitudinal axis, a proximal end portion and a distal end portion configured for insertion into a patient. The cannula tube may have an internal lumen extending from the proximal end portion to the distal end portion for receiving an obturator therein. The cannula tube may also have a housing coupled to the cannula tube between the proximal and distal ends of the cannula tube so as to be positioned within the patient when the distal end of the cannula tube is inserted into the patient. The housing may be movable relative to the cannula tube between a closed position and an open position. In addition, the housing may include a light source and an image sensor configured to provide a first image data stream of the patient's anatomy and the surgical tool when the housing is in the open position within the patient.

The surgical system may also include, in various embodiments, a second imaging device configured for insertion into a patient and to provide a second image data stream of the patient's anatomy and the surgical tool. This second imaging device may be a cannula assembly similar to the hereinabove-described cannula assembly, or the second imaging device may be any other type of imaging device, e.g., a traditional endoscope or laparoscope, typically employed during surgical procedures. The surgical system may also include an image processor configured to receive the first and second image data streams and to process the first and second image data streams into a combined image data stream, e.g., a stereoscopic or 3D image stream, of the patient's anatomy and the surgical tool. Advantageously, the system may also include a display device on which is displayed to a user, e.g., a surgeon, the combined image data stream.

Still further, the surgical system may include, in accordance with various embodiments thereof, a spatial processor configured to determine, based upon the combined image data stream of the patient's anatomy and the surgical tool, positional data relative to the patient's anatomy and the surgical tool. Advantageously, this positional data may be real-time positional data such that the relative positions of the surgical tool and the patient's anatomy are continuously being updated throughout the course of the surgical procedure. The surgical system may also include a navigation processor configured to generate and provide, based upon the real-time positional relationship data between the patient's anatomy and the surgical tool, navigation data to the user on the display device.

In accordance with various embodiments, the surgical system may also be configured such that the real-time positional data relating to the patient's anatomy and the surgical tool includes current positional data of the surgical tool relative to the patient's anatomy. Still further, the navigation data that the navigation processor is configured to generate may be based upon a desired engagement position of the surgical tool relative to the patient's anatomy, e.g., a position at which the surgical tool is able to engage the patient's tissue for its intended task, e.g., stapling the tissue. The navigation data generated by the navigation processor may be in the form of a navigation path of the surgical tool relative to the patient's anatomy. Advantageously, the navigation processor may be configured to display the preferred navigation path on the display device with a label that conveys to the user that it is safe for the surgical tool to be moved along the navigation path.

In various embodiments, any one or more of the image processor, the spatial processor and/or the navigation processor may be incorporated into the cannula assembly. Additionally or alternatively, any one or more of the image processor, the spatial processor and/or the navigation processor may be incorporated into a control device that is external to the cannula assemblies. In such an embodiment, the control device that is external to the cannula assemblies may be configured to transmit and/or receive one or more of wired or wireless data signals from and/or to the cannula assemblies.

In still further embodiments, there is provided a cannula assembly for performing a surgical procedure on a patient. The cannula assembly may include a cannula tube having a longitudinal axis, a proximal end portion and a distal end portion configured for insertion into a patient. The cannula tube may have a housing coupled thereto so as to be positioned within the patient when the distal end of the cannula tube is inserted into the patient. The housing may be movable relative to the cannula tube between a closed position and an open position. The housing may include a light source and an image sensor configured to provide a first image data stream of the patient's anatomy and a surgical tool when the housing is in the open position within the patient. A processor may be configured to the process the first data image stream and to generate navigation data based on the image data stream.

According to various such embodiments, the processor may be configured to receive a second image data stream from a second imaging device, e.g., either a similar cannula assembly or a traditional endoscope of laparoscope, that provides the second image data stream of the patient's anatomy and the surgical tool. The processor may be configured to process the first and second image data streams into a combined image data stream of the patient's anatomy and the surgical tool. The processor may further be configured to display the combined image data stream to a display device for viewing by a user.

Still further, the processor may be configured to generate, based upon the combined image data stream of the patient's anatomy and the surgical tool, real-time positional data relative to the patient's anatomy and the surgical tool. The positional data generated by the processor from the combined image stream may include current position data of the surgical tool and the patient's anatomy, and/or it may include desired engagement position data of the surgical tool and the patient's anatomy. In this way, the navigation data generated by the navigation processor may relate to a preferred navigation path of the surgical tool relative to the patient's anatomy from the current position to the desired engagement position. In various embodiments, the processor may be configured to display the preferred navigation path on the display device with a label that conveys to the user that it is safe for the surgical tool to be moved along the navigation path.

According to the various aspects, the processor may be incorporated into the cannula assembly, or may be external to the cannula assembly. Additional or alternatively, the processor may include various controller devices, some of which are internal relative to the cannula assembly and some of which are external relative to the cannula assembly, these various controller devices operating to perform, either separately or together, the various operations described herein. Of course, it will be recognized that, multiple different processors may be employed to perform the various operations, there being no limit on the number or configuration of processors that may be employed.

Among various other advantages provided by certain embodiments as will be evident from the Detailed Description below, there may also be the benefit that fewer punctures through a patient, e.g., through an abdominal wall or other bodily surface, are made during a surgical procedure. As set forth above, a surgeon typically performs a laparoscopic procedure using multiple cannulas inserted through individual incisions, wherein at least one such cannula and incision is occupied by an illumination/imaging device, such as a traditional endoscope and/or laparoscope. According to various embodiments thereof, there may be provided a cannula assembly and/or system therefor that eliminates the need for this separate puncture by a cannula assembly for an endoscope/laparoscope, since it provides, in certain embodiments, a cannula assembly which provides both an illumination/imaging device (e.g., mounted or coupled to the cannula tube) and an internal lumen through which a separate surgical tool (e.g., a surgical stapler, etc.) may be inserted. The reduction of at least one puncture during a surgical procedure, as may be enabled in certain embodiments, may improve the safety of the surgical procedure by avoiding potential complications, reducing pain and/or speeding the patient's recovery.

### BRIEF DESCRIPTION OF THE DRAWINGS

FIG. 1 is a system block diagram that illustrates cannula assemblies employed in a surgical procedure, in accordance with various embodiments.

FIG. 2 shows a block diagram illustrating an example of a device controller in accordance with various embodiments.

FIG. 3 shows a block diagram illustrating an example of an imaging controller for a system in accordance with embodiments.

### DETAILED DESCRIPTION

Generally, there is provided hereinbelow imaging systems and, more particularly, endoscopic imaging systems. In various embodiments, and as will be set forth in detail below, there may be provided cannula assemblies for use in a surgical system that provides real-time navigational guidance to a surgeon during a surgical procedure, enabling on-the-fly adjustments to the navigational guidance based on continuously updated positional data obtained by the cannula imaging devices.

Reference will now be made in detail to specific embodiments illustrated in the accompanying drawings. In the following detailed description, numerous specific details are set forth to provide a thorough understanding of the disclosure. However, it will be apparent to one of ordinary skill in the art that embodiments may be practiced without these specific details. In other instances, known methods, procedures, components, circuits, and networks have not been described in detail so as not to unnecessarily obscure aspects of the embodiments.

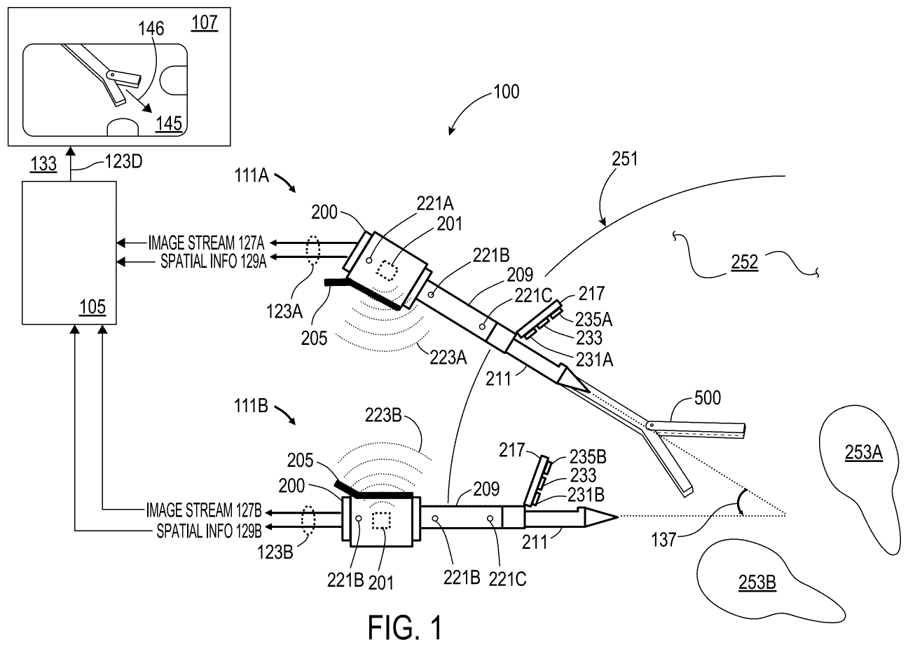

FIG. 1 shows a surgical system 100 illustrating an example embodiment. In this embodiment, there is shown a surgical system 100 in which there are two cannula assemblies 111A, 111B. Although FIG. 1 illustrates two such cannula assemblies, it should be understood that certain advantages may be obtained with a single such cannula assembly. This embodiment having two cannula assemblies will have additional advantages as shown and described below.

In the embodiment shown in FIG. 1, each of the cannula assemblies 111A, 111B include a housing 200, a device controller 201, an actuator handle 205, a cannula tube 209, an obturator 211, and a sensor housing 217. Although not shown herein, it should be understood by those skilled in the art that the cannula assembly 111A (and other cannula assemblies shown and described herein) may include other components and features in addition to those described herein. For example, any of the herein-described cannula assemblies 111A, 111B may include sealing components, such as an instrument seal for sealing around an instrument inserted therethrough, a zero seal for sealing the cannula assembly in the absence of any instrument inserted therethrough, and/or any number of different ports, e.g., insufflation or irrigation ports, for the introduction of various gases or liquids into the surgical site.

The cannula tubes 209 may be formed of a variety of cross-sectional shapes. For example, the cannula tubes 209 can have a generally round or cylindrical, ellipsoidal, triangular, square, rectangular, and D-shaped (in which one side is flat). The cannula tube 209 may include an internal lumen 202 into which the obturator 211 is inserted. The obturator 211 can be retractable and/or removable from the cannula tube 209. In some embodiments, the obturator 211 is made of solid, non-transparent material. In another embodiment, all or parts of the obturator 211 are made of optically transparent or transmissive material such that the obturator 211 does not obstruct the view through the camera (discussed below). The obturator 211 may have a tip shape that is configured to penetrate, either via incision or via insertion between tissue planes, through the abdominal wall 251 of the patient.

The sensor housing 217 can be integral with the cannula tube 209 or it may be formed as a separate component that is coupled to the cannula tube 209. In either case, the sensor housing 217 can be disposed on or coupled to the cannula tube 209 at a position proximal to the distalmost end of the cannula tube 209 such that it is positioned within the patient's body when the distal end portion of the cannula tube 209 has been inserted into the patient. In some embodiments, the sensor housing 217 can be actuated by the actuator handle 205 to open, for example, after being inserted into the patient's 117 body cavity 252. The sensor housing 217 can reside along cannula tube 209 in the distal direction such that it is positioned within the body cavity 252 of a patient (e.g., patient 117) during a surgical procedure. At the same time, the sensor housing 217 can be positioned proximal to the distal end such that it does not interfere with the insertion of the distal end of the cannula tube 209 as it is inserted into a patient (e.g., patient 117).

In some embodiments, the sensor housings 217 may include one or more image sensors 231A, 231B and a light source 235A, 235B. The light sources 235A, 235B may be dimmable light-emitting device, such as a LED, a halogen bulb, an incandescent bulb, or other suitable light emitter. The image sensors 231A, 231B may be devices configured to detect light reflected from the light source 235 and output an image signal. The image sensors 231A, 231B can be, for example, a charged coupled device (“CCD”) or other suitable imaging sensor. In some embodiments, the image sensors 231A, 231B includes at least two lenses providing stereo imaging. In some embodiments, the image sensors 231A, 231B can be an omnidirectional camera. The image data based on image signals generated by image device 231A can eventually be overlaid onto the image data based on image signals generated by image device 231B, or vice versa, so as to provide a combined image stream, as will be described more fully below.

The cannula tube 209, the obturator 211, and the sensor housing 217 of the individual cannula assemblies 111A, 111B can be inserted into the body cavity 252 of a patient (e.g., patient 117) and positioned relative to each other, e.g., such as at an angle 137 with respect to each other, so as to provide differing fields-of-view from the sensor housing 217 of surgical tools, e.g., surgical stapler 500, and patient anatomical features, e.g., patient anatomical features 253A, 253B, within the body cavity 252 of the patient 117, as will be described in additional detail below.

The device controller 201 may be one or more devices that process signals and data to generate respective image streams 127A, 127B and spatial information 129A, 129B of the cannula assemblies 111A, 111B. Spatial data, e.g., spatial information 129A, 129B, is data that relates to the relative position of various components, in this case the cannula assembles 11A, 111B. Various different spatial data components are contemplated. For example, in the embodiment shown, the cannula assemblies 111A, 111B may include spatial data components, e.g., in the form of antennas 221A, 221B, 221C. In some embodiments, the device controller 201 can determine the spatial information 129A, 129B by processing data from spatial sensors (e.g., accelerometers) to determine the relative position, angle, and rotation of the cannula assemblies 111A, 111B. In some embodiments, the device controller 201 can also determine the spatial information 129A, 129B by processing range information received from sensors (e.g., image sensor 231 and LiDAR device 233) in the sensor housing 217. Additionally, in some embodiments, the device controller 201 can process the spatial information 129A, 129B by processing signals received via the antennas 221A, 221B, 221C to determine relative distances of the cannula assemblies 111A, 111B. It is understood that, in some embodiments, less than all, e.g., only one or none, of the cannula assemblies 111A, 111B provides spatial information 129. However, the spatial information, as shown and described herein, is advantageous to ensure that image streams are accurately combined relative to each other.

As mentioned above, in some embodiments, the sensor housing 217 can include a LiDAR device 233. The LiDAR device 233 can include one or more devices that illuminate a region with light beams, such as lasers, and determine distance by measuring reflected light with a photosensor. The distance can be determined based on a time difference between the transmission of the beam and detection of backscattered light. For example, using the LiDAR device 233, the device controller 201 can determine spatial information 129 by sensing the relative distance and rotation of the cannulas 209 or the sensor housing 217 inside a body cavity.

Additionally, where antennas are employed, the antennas 221A, 221B, 221C can be disposed along the long axis of the cannula assemblies 111A, 111B. In some embodiments, the antennas 221A, 221B, 221C can be placed in a substantially straight line on one or more sides of the cannula assemblies 111A, 111B. For example, two or more lines of the antennas 221A, 221B, 221C can be located on opposing sides of the housing 203 and the cannula tube 209. Although FIG. 1 shows a single line of the antennas 221A, 221B, 221C on one side of the cannula assemblies 111A, 111B, it is understood that the additional lines of the antennas 221A, 221B, 221C can be placed in opposing halves, thirds, or quadrants of the cannula assemblies 111A, 111B.

As illustrated in FIG. 1, in some embodiments, the device controllers 201 can transmit a ranging signal 223. In some embodiments, the location signals are ultra-wideband (“UWB”) radio signal usable to determine a distance between the cannula assemblies 111A, 111B less than or equal to 1 centimeter based on signal phase and amplitude of the radio signals, as described in IEEE 802.15.4Z. The device controller 201 can determine the distances between the cannula assemblies 111A, 111B based on the different arrival times of the ranging signals 223A and 223B at their respective antennas 221A, 221B, 221C. For example, referring to FIG. 1, the ranging signal 223A emitted by cannula assembly 111A can be received by cannula assembly 111B at antenna 221C and an amount of time (T) after arriving at antenna 221B. By making a comparison of the varying times of arrival of the ranging signal 223A at two or more of the antennas 221A, 221B, 221C, the device controller 201 of cannula assembly 111B can determine its distance and angle from cannula assembly 111A. It is understood that the transmitters can be placed at various suitable locations within the cannula assemblies 111A, 111B. For example, in some embodiment, the transmitters can be located in the cannulas 209 or in the sensor housings 217.

In some embodiments, the spatial sensors 317 can include one or more of, piezoelectric sensors, mechanical sensors (e.g., a microelectronic mechanical system (“MEMS”), or other suitable sensors for detecting the location, velocity, acceleration, and rotation of the cannula assemblies (e.g., cannula assemblies 111A, 111B).

As set forth above, FIG. 1. also illustrates a surgical tool, e.g., in this case a surgical stapler 500. Of course, it should be recognized that the surgical tool 500 may be any conceivable type of surgical tool, depending on the surgical procedure being performed. Additionally, and as also set forth above, FIG. 1. illustrates patient anatomical features, e.g., patent anatomical features 253A, 253B. Again, it should be recognized that the patent anatomical features 253A, 253B may be any conceivable anatomical features, depending on the location of the body at which the surgical procedure is being performed.

In some embodiments, the cannula assemblies 111A, 111B may also include a processor or device controller 201 that is configured to receive the image data from the image sensors 231A, 231B and to perform certain processing steps regarding the image data prior to its being displayed on a separate display device, e.g., display device 107 having a display 145. The processor or device controller 201 can be a computing device connecting the cannula assemblies 111A, 111B to the display 107, e.g., either directly or indirectly via additional processors, through one or more wired or wireless communication channels 123A, 123B. In the embodiment shown in FIG. 1, the system 100 also includes such an additional processor, e.g., an imaging/navigation controller 105, that performs additional processing steps, as will be described in further detail below. The imaging/navigation controller 105 may also be a computing device that is connected to the display device 107 and the cannula assembly 111A through the one or more wired or wireless communication channels 123A, 123B.

The communication channels 123A, 123B may use various serial, parallel, video transmission protocols suitable for their respective signals such as image streams 127A, 127B and processed image stream 133. The imaging/navigation controller 105 can include hardware, software, or a combination thereof for performing operations. The display device 107 can be a liquid crystal display (LCD) display, organic light emitting diode displays (OLED), cathode ray tube display, or other suitable display device. In some embodiments, the display device 107 can be a stereoscopic head-mounted display, such as a virtual reality headset.

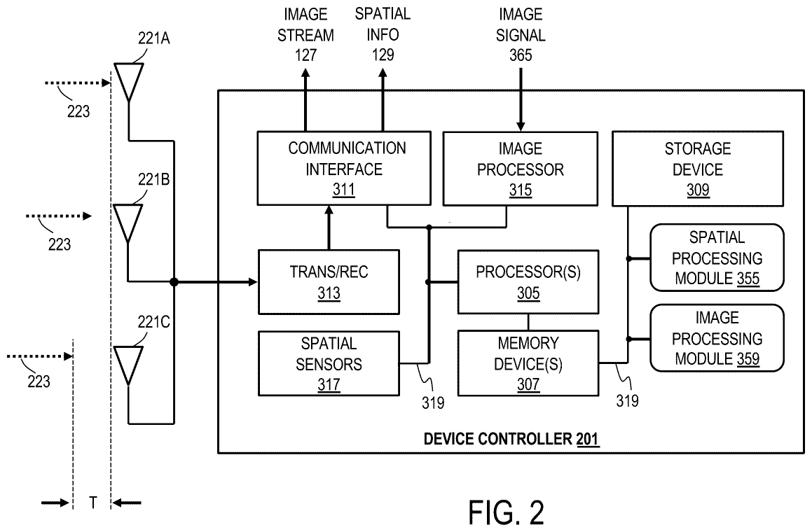

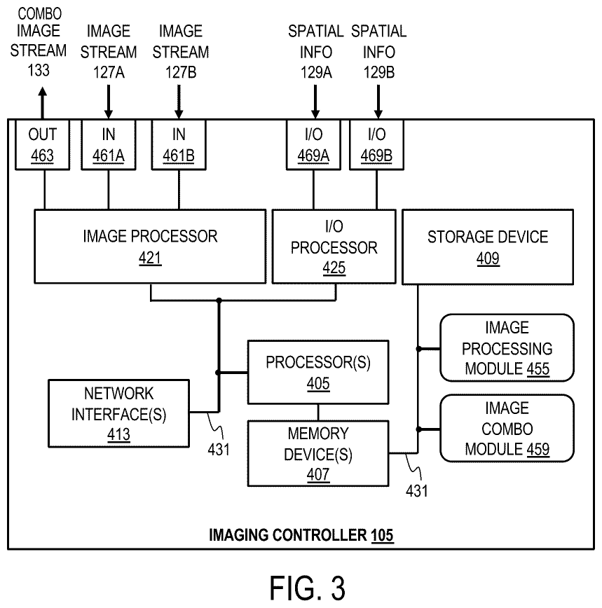

It should be noted that, while the description hereinbelow describes various components, operations and functions as potentially being present or performed by one or either of the device controller 201 and/or the image/navigation controller 105, it is contemplated that the below-described components, operations and functions may be present or performed entirely in a single one of the device controller 201 or the image/navigation controller 105, that additional controllers/processor may be present that perform any one or more or portions of said operations or functions, and/or that the components described herein may be shared across the device controller 201 and the image/navigation controller 105 (and/or such additional processors) such that the device controller 201 and the image/navigation controller 105 may share responsibility for performing any one or more of the herein-described operations or functions. As will be shown below, FIGS. 2 and 3 illustrate an embodiment in which device controller 201 has components for, and performs, certain operations and functions, while the image/navigation controller 105 has components for, and performs, certain operations and functions. It should be recognized by those skilled in the art that, in accordance with other embodiments thereof, there may be included processors either internal or external to the cannula assemblies 111A, 111B for performing these operations and functions, and that, although described in connection with a certain processor, there is no intent to limit to any particular structure or location of such components, operations or functions. The example embodiment described hereinbelow is merely one way that such processor may be employed.

In operation, and in accordance with an example embodiment as mentioned above, the image sensors 231A, 231B generate image signals relating to the body cavity 252 of the patient, including image signals relating to, e.g., surgical tool 500 and patient anatomical features 253A, 253B. These image signals are processed by the device controller 201 to generate respective image streams 127A, 127B relating thereto.

Simultaneously, any one or more of the spatial data devices, e.g., antennas 221A, 221B, 221C or LiDAR device 233 etc., may generate spatial data relating to the cannula assemblies 111A, 111B. This spatial data may be received by and processed by the device controller 201 to generate respective spatial information 129A, 129B relating thereto. The image data streams 127A, 127B and/or the spatial information 129A, 129B may then be used, e.g., via the device controller 201 and/or the image/navigation controller 105, to generate stereoscopic image data of the surgical tool 500 and the patient anatomical features 253A, 253B within the body cavity 252. Advantageously, the stereoscopic image data of the surgical tool 500 and the patient anatomical features 253A, 253B may be utilized by the system 100 to provide a 3D display to the surgeon on the display device 107.

In addition to using the stereoscopic image data of the surgical tool 500 and the patient anatomical features 253A, 253B to provide a 3D display to the surgeon on the display device 107, the imaging/navigation controller 105 may also use the stereoscopic image data of the surgical tool 500 and the patient anatomical features 253A, 253B to generate current spatial, or positional, data of the surgical tool 500 and of the patient anatomical features 253A, 253B. Specifically, the stereoscopic image data of the surgical tool 500 and the patient anatomical features 253A, 253B enable the imaging/navigational processor 105 to calculate current positional data that represents where the surgical tool 500 and the patient anatomical features 253A, 253B are currently located within the body cavity 252.

Still further, the imaging/navigation controller 105 may then be employed to determine a desired engagement position data of the surgical tool 500 relative to the patient anatomical features, 253A, 253B. The desired engagement position of the surgical tool relative to the patient anatomical features may be, for example, a position at which the surgical tool 500 will be engaged with the patient anatomical features 253A, 253B for the purpose of conducting its intended surgical task. By way of example, referring to the surgical stapler 500 depicted in FIG. 1, the desired engagement position may be the position at which the surgical stapler 500 will be engaged with patient tissue 253A in order to grasp, compress and fire staples therethrough. Of course, it should be understood by persons of skill in the art that the desired engagement position may be any conceivable position depending on the type of the surgical tool being used in the surgical procedure, the particular type of patient tissue to be engaged during the surgical procedure, the particular surgical task desired to be performed, the unique physical characteristics of the patient, etc. The imaging/navigation processor 105 may be configured to receive, whether via imaging sensors 231A, 231B or via user inputs (described in additional below in connection with, e.g., the I/O processors 425 in FIG. 3) or via stored data memory locations (also described in additional detail below in connection with, e.g., the storage device 409 I FIG. 3), such data about any or all of these factors and may utilize such data in determining the desired engagement position data.

Still further, the imaging/navigation controller 105 may then be employed to compare the current positional data of the surgical tool 500 and of the patient anatomical features 253A, 253B to the desired engagement position data of the surgical tool 500 relative to the patient anatomical features, 253A, 253B. If the current position of the surgical tool 500 and of the patient anatomical features 253A, 253B is the same as the desired engagement position, then the surgical tool 500 is in position to perform its given surgical task, and the surgical tool 500 need not be moved relative to the patient anatomical features 253A, 253B. Rather, the surgical tool 500 may be actuated, e.g., in the case of a surgical stapler, it may be clamped and fired on the tissue intended to be stapled.

If the current position of the surgical tool 500 and of the patient anatomical features 253A, 253B is not the same as the desired engagement position, then the surgical tool 500 is not in position to perform its given surgical task, and the surgical tool 500 does need to be moved relative to the patient anatomical features 253A, 253B. In this case, in accordance with various embodiments, the imaging/navigation controller 105 may use the current positional data and the desired engagement position data of the surgical tool 500 and patient anatomical features 253A, 253B to generate navigational path data. More specifically, the imaging/navigation controller 105 may use the current positional data and the desired engagement position data of the surgical tool 500 and patient anatomical features 253A, 253B to generate a navigational path via which the surgical tool 500 may be moved between its current position relative to a patient anatomical feature 253A, and its desired engagement position relative to the patient anatomical feature 253A. Continuing with the example described hereinabove, the imaging/navigation controller 105 may use data relating to the current and desired positions of the surgical tool 500 relative to the patient anatomical feature 253A to generate a navigational path along which the surgical tool 500 may be manipulated by the surgeon in order for the surgical tool 500 to be moved from its current position relative to a patient anatomical feature 253A to its desired engagement position relative to the patient anatomical feature 253A. As mentioned above, it should be understood by persons of skill in the art that the navigation path data may refer to any conceivable path depending on the type of the surgical tool being used in the surgical procedure, the particular type of patient tissue to be engaged during the surgical procedure, the particular surgical task desired to be performed, the unique physical characteristics of the patient, etc. And, as above, the imaging/navigation processor 105 may be configured to utilize any or all of such types of data in determining the navigation path data.

In still further embodiments thereof, it is also contemplated that the imaging/navigation controller 105 may compare the generated navigation path data to stored safety data to determine whether the navigation path data is safe for the surgeon to move the surgical tool therebetween. The stored safety data may be any type of stored data that relates to the safety of the surgical procedure being performed. For example, the stored safety data may consist of safety data relating to the particular surgical tool 500 being employed, e.g., in the case of a surgical stapler, the stored safety data may relate to optimal clamping angles for the surgical stapler, preferred stapler lengths or staple configurations, preferred tissue thickness ranges across which a stapler can be fired, or any other conceivable safety information that would be useful for a surgeon to know as the surgical procedure is being conducted. Other types of stored safety data may also be employed, e.g., the stored safety data may consist of safety data relating to the patient's anatomy, such as preferred tissue thickness ranges across which a surgical stapler can be fired, or may consist of data related to known anatomical structures, e.g., vasculature or major arteries, that should not be stapled across, or may consist of anatomical feature data that is patient-specific. Of course, any other conceivable safety information that would be useful for a surgeon to know as the surgical procedure is being conducted, may be employed in various embodiments.

If the imaging/navigation controller 105 determines that it is safe for the surgeon to move the surgical tool 500 between the current position and the desired engagement position via the generated navigation path, the imaging/navigation controller 105 may also generate a navigation path label 146. Advantageously, the imaging/navigation controller 105 may generate the navigation path label 146 in the form of a symbol or text that, upon being viewed by the surgeon on the display 145 of the display device 107, lets the surgeon know that the generated navigation path is safe for the surgeon to move the surgical tool 500 along. This symbol or text may be any conceivable words or symbols, or set of words or symbols, that is capable of conveying the desirability of the path to be taken, for example, a line, an arrow, or a pointer, etc. that illustrates the navigation path to be taken. In some embodiments, the symbol or text could be displayed with a particular color, e.g., green, to further convey that the path is a safe one along which the surgical tool 500 may be moved.

Additionally or alternatively, the imaging/navigation controller 105 could be configured to determine and display non-preferred or unsafe navigational paths. For example, if the imaging/navigation controller 105 determines that it is unsafe for the surgeon to move the surgical tool 500 between the current position and the desired engagement position via the generated navigation path, the imaging/navigation controller 105 may generate a different type of the navigation path label 146. Specifically, the imaging/navigation controller 105 may generate the navigation path label 146 in the form of a symbol or text that, upon being viewed by the surgeon on the display 145 of the display device 107, lets the surgeon know that the generated navigation path is unsafe for the surgeon to move the surgical tool 500 along. As above, this symbol or text may be any conceivable words or symbols, or set of words or symbols, that is capable of conveying a path that should not be taken, for example, a line, an arrow, or a pointer, etc. that is red in color.

In various embodiments thereof, it is contemplated that the various different operation steps, such as those described hereinabove, can be performed numerous times over the course of a surgical procedure. Optimally, for example, it is contemplated that the various different operation steps described hereinabove can be performed continuously over the course of a surgical procedure, allowing real-time imaging and on-the-fly adjustments to the navigational guidance during the surgical procedure. In such an embodiment, the image streams 127A, 127B can generate constantly-updated image data relating to the surgical tool 500 and the patient anatomical features 253A, 253B, thereby allowing the processors, e.g., either or both of the device controller 201 and/or the imaging/navigation controller 105, to constantly update the positional data relating to the surgical tool 500 and the patient anatomical features 253A, 253B. In this way, the processors can take into account the movement of the surgical tool 500 and, significantly, the movement of the patient anatomical features 253A, 253B, during the surgical procedure so as to adjust, if needed, the navigation determinations made thereby.

Such an arrangement, e.g., wherein the various different operation steps described hereinabove are performed continuously over the course of a surgical procedure so as to allow real-time imaging and on-the-fly adjustments to the navigational guidance during the surgical procedure, provides significant advantages over surgical systems that lack such functionality. For example, typical prior art surgical systems may rely on pre-operative tissue models to provide positional data relating to the patient anatomical features 253A, 253B within a patient's body cavity. These pre-operative tissue models use imaging or positional data that is determined before a surgical procedure takes place, and thus do not account for any movement of the tissues that can take place during the surgical procedure. In contrast, the systems and methods described hereinabove may improve upon this by continuously updating the positional data of the tissue as that tissue is moved within the body cavity, e.g., when it is moved by gravity, or when it is moved by pressure provided by insufflation gases within the body cavity, or when the surgeon moves it to provide greater access to certain areas of the surgical site, etc. Among the advantages of such functionality is the ability of the system to provide “best approach”-type navigational data, whereby the navigational path provided to the surgeon on the display device 107 is not merely the best navigational approach at a single previous moment of time (e.g., a pre-surgical moment before the surgical procedure has begun), but the best navigational approach is constantly being checked during the course of the surgical procedure and continuously adjusted, if needed, so that the navigational approach isn't relying on positional data that has changed.

FIG. 2 shows a functional block diagram illustrating an example of a device controller 201 in accordance with aspects thereof. It is noted, as mentioned above, that the device controller 201 shown and described herein is merely representative of various possible equivalent-computing devices that can perform the processes and functions described herein. To this extent, in some embodiments, the functionality provided by the device controller 201 can be any combination of general and/or specific purpose hardware and/or program instructions. In each embodiment, the program instructions and hardware can be created using standard programming and engineering techniques. In the embodiment shown, the device controller 201 may include a processor 305, a memory device 307, a storage device 309, a communication interface 311, a transmitter/receiver 313, an image processor 315, spatial sensors 317, and a data bus 319.

In various embodiments, the processor 305 may include one or more microprocessors, microchips, or application-specific integrated circuits. The memory device 307 may include one or more types of random-access memory (RAM), read-only memory (ROM) and cache memory employed during execution of program instructions. The processor 305 may use the data buses 319 to communicate with the memory device 307, the storage device 309, the communication interface 311, the image processor 315, and the spatial sensors 317. The storage device 309 may comprise a computer-readable, non-volatile hardware storage device that stores information and program instructions. For example, the storage device 309 can be one or more, flash drives and/or hard disk drives. The transmitter/receiver 313 can be one or more devices that encodes/decodes data into wireless signals, such as the ranging signal 223.

The processor 305 executes program instructions (e.g., an operating system and/or application programs), which can be stored in the memory device 307 and/or the storage device 309. The processor 305 may also execute program instructions of a spatial processing module 355 and an image processing module 359. The spatial processing module 335 can include program instructions that determine the spatial information 129 by combining spatial data provided from the transmitter/receiver 313 and the spatial sensors 317. The image processing module 359 can include program instructions that, using the image signals 365 from the imaging sensors 231A, 231B register and overlay the images to generate the image streams 127A, 127B. The image processor 315 can be a device configured to receive an image signal 365 from an image sensor (e.g., image sensors 231A, 231B) and condition images included in the image signal 365. In accordance with aspects thereof, conditioning the image signal 365 can include normalizing the size, exposure, and brightness of the images. Also, conditioning the image signal 365 can include removing visual artifacts and stabilizing the images to reduce blurring due to motion. Additionally, the image processing module 359 can identify and characterize structures in the images.

FIG. 3 shows a functional block diagram illustrating an imaging and navigation controller 105 in accordance with aspects thereof. It is noted, as mentioned above, that the imaging/navigation controller 105 shown and described herein is merely representative of various possible equivalent-computing devices that can perform the processes and functions described herein. To this extent, in some embodiments, the functionality provided by the imaging/navigation controller 105 can be any combination of general and/or specific purpose hardware and/or program instructions, and the program instructions and hardware can be created using standard programming and engineering techniques. The imaging/navigation controller 105 may include, e.g., a processor 405, a memory device 407, a storage device 409, a network interface 413, an image processor 421, an I/O processor 425, and a data bus 431. Also, the imaging/navigation controller 105 can include image input connections 461A, 461B, image output connection 463 that receive and transmit image signals from the image processor 421. Further, the imaging and navigation controller 105 can include input/output connections 469A, 469B that receive/transmit data signals from the I/O processor 425.

In embodiments, the imaging and navigation controller 105 can include one or more microprocessors, microchips, or application-specific integrated circuits. The memory device 407 can include one or more types of random-access memory (RAM), read-only memory (ROM) and cache memory employed during execution of program instructions. Additionally, the imaging and navigation controller 105 can include one or more data buses 431 by which it communicates with the memory device 407, the storage device 409, the network interface 413, the image processor 421, and the I/O processor 425. The storage device 409 can comprise a computer-readable, non-volatile hardware storage device that stores information and program instructions. For example, the storage device 409 can be one or more, flash drives and/or hard disk drives. The storage device 409 may store any type of useful data. For example, in various embodiments, it may store, as set forth previously, data relating to the type of the surgical tool being used in the surgical procedure, the particular type of patient tissue to be engaged during the surgical procedure, the particular surgical task desired to be performed, the unique physical characteristics of the patient, etc. Still further, the storage device 409 may store data relating to safety data, for example, as set forth previously, safety data relating to the particular surgical tool 500 being employed (e.g., in the case of a surgical stapler, safety data relating to optimal clamping angles for the surgical stapler, preferred stapler lengths or staple configurations), safety data relating to the patient's anatomy (e.g., such as preferred tissue thickness ranges across which a surgical stapler can be fired, data related to known anatomical structures like vasculature or major arteries that should not be stapled across, etc.) or any other conceivable type of safety information.

The I/O processor 425 can be connected the processor 405 and can include any device that enables an individual to interact with the processor 405 (e.g., a user interface) and/or any device that enables the processor 405 to communicate with one or more other computing devices using any type of communications link. For example, the I/O processor 425 may include any type of device that enables a surgeon to input information useful to the surgical procedure. For example, it may allow to be inputted information relating to, e.g., the type of the surgical tool being used in the surgical procedure, the particular type of patient tissue to be engaged during the surgical procedure, the particular surgical task desired to be performed, the unique physical characteristics of the patient, safety data relating to the particular surgical tool 500 being employed, safety data relating to the patient's anatomy, or any other conceivable type of safety information. In various embodiments, the I/O processor 425 can generate and receive, for example, digital and analog inputs/outputs according to various data transmission protocols.

The processor 405 executes program instructions (e.g., an operating system and/or application programs), which can be stored in the memory device 407 and/or the storage device 409. For example, the processor 405 may be employed, in various embodiments, to generate the herein above-referenced current positional data. More specifically, as and set forth above, the processor 405 may use the stereoscopic image data of the surgical tool 500 and the patient anatomical features 253A, 253B to generate current spatial, or positional, data that represents where the surgical tool 500 and the patient anatomical features 253A, 253B are currently located within the body cavity 252.

In still further embodiments, the processor 405 may be employed to, as set forth above, determine desired engagement position data, e.g., a position at which the surgical tool 500 will be engaged with the patient anatomical features 253A, 253B for the purpose of conducting its intended surgical task. The processor 405 may be configured to generate this desired engagement position data by processing other data (e.g., the type of surgical procedure, the type of surgical tool being used etc.) received from one or more different data sources (e.g., from the imaging sensors 231A, 231B, from the user inputs of the I/O processors, and/or from stored data memory locations such as the storage device 409. The processor 405 may also be employed to, as set forth above, compare the current positional data of the surgical tool 500 and of the patient anatomical features 253A, 253B to the desired engagement position, and if the current position is not the same as the desired engagement position, to generate the navigational path for moving the surgical tool 500 to its desired engagement position relative to the patient anatomical features 253A, 253B. Still further, the processor 405 may also be employed to, as previously described, compare the generated navigation path data to stored safety data (e.g., stored safety data that may be stored for example in storage device 409) to determine that the navigation path data is safe for the surgeon to move the surgical tool therebetween. In addition, the processor 405 may be employed, as described hereinabove, to generate the navigation path label 146 in the form of a symbol or text that lets the surgeon know that the generated navigation path is safe for the surgeon to move the surgical tool 500 along.

In various embodiments, and as set forth above, the processor 405 may be configured to perform these different operation steps numerous times, and optimally to perform them continuously, over the course of the surgical procedure. In this way, the processor 405 obtains image data from the image sensors 231A, 231B in real-time, enabling the navigational guidance to be adjusted on-the-fly based on up-to-date positional data relating to the surgical tools 500 and the patient anatomical features 253A, 253B. In this way, and unlike systems. That rely solely on pre-operative tissue models, the processors can take into account the movement of the surgical tool 500 and, significantly, the movement of the patient anatomical features 253A, 253B, during the surgical procedure so as to adjust, if needed, the navigation guidance made thereby.

The processor 405 can also execute program instructions of an image processing module 455 and an image combination module 459. The image processing module 455 can be configured to stabilize the images to reduce the blurring, compensate for differences in tilt and rotation, remove reflections and other visual artifacts from the images, and normalize the images. Additionally, the image processing module 455 can be configured to identify and characterize structures, such as surgical tools 500 and/or tissues 253A, 253B, in the images. Further, the imaging processing module can be configured to determine obstructions in the overlapping fields of view and process the images streams 127A, 127B to remove the obstructions, if desirable.

The image combination module 459 can be configured to analyze images received in image streams 127A, 127B from the cannula assemblies and overlay them into a single, combined image stream 133 based on the spatial information. In some embodiments, the image combination module 459 generates the combined image stream 133 by registering and overlaying the image stream 127A, 127B based on the respective fields-of-view of the cannula assemblies. In some embodiments, either of the cannula assemblies can be selected by an operator (e.g., via I/O processor 425) as a primary cannula assembly (e.g., cannula assembly 111A), and the image combination module 459 can generate the combined image stream 133 by using the image stream 127B of the secondary cannula assembly to augment the image stream 127A. The combined image stream 133 can also provide a 3D view from the perspective of the primary cannula assembly or vice versa). In some embodiments, the combined image stream 133 lacks certain obstructions removed by the image processing module 455.

The image processing module 421 may, in accordance with various embodiments, operate to generate and display the navigation path label 146. As set forth above, the navigation path label 146 may be any type of symbol or text that lets the surgeon know that the generated navigation path is safe for the surgeon to move the surgical tool 500 along. The image processing module 421 may generate the appropriate label and provide data relating thereto to the image combination module 459 so that the navigation path label 146 may be accurately combined into the combined image stream 133 along with the other image streams 127A. 127B.

The system and methods described hereinabove are not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope thereof, in addition to those enumerated herein, will be apparent to those skilled in the art from the foregoing descriptions. Such modifications and variations are intended to fall within the scope of the appended claims. The systems and methods described hereinabove are to be limited only by the terms of the appended claims, along with the full scope of equivalents to which such claims are entitled. It is also to be understood that the terminology used herein, e.g., “and”, “or”, “including”, “at least” as well as the use of plural or singular forms, etc., is for the purpose of describing examples of embodiments and is not intended to be limiting.

1. A surgical system for performing a surgical procedure on a patient using a surgical tool, comprising:
a cannula assembly including a cannula tube having a longitudinal axis, a proximal end portion and a distal end portion configured for insertion into a patient, the cannula tube having an internal lumen extending from the proximal end portion to the distal end portion, the cannula tube having a housing coupled to the cannula tube between the proximal and distal ends of the cannula tube so as to be positioned within the patient when the distal end of the cannula tube is inserted into the patient, the housing movable relative to the cannula tube between a closed position and an open position, the housing including a light source and an image sensor configured to provide a first image data stream of a patient's anatomy and the surgical tool when the housing is in the open position within the patient;
a second imaging device configured for insertion into a patient and to provide a second image data stream of the patient's anatomy and the surgical tool;
an image processor configured to receive the first and second image data streams and to process the first and second image data streams into a combined image data stream of the patient's anatomy and the surgical tool;
a display device to display the combined image data stream to a user;
a spatial processor configured to determine, based upon the combined image data stream of the patient's anatomy and the surgical tool, real-time positional data relative to the patient's anatomy and the surgical tool; and
a navigation processor configured to generate and provide, based upon the real-time positional relationship data between the patient's anatomy and the surgical tool, navigation data to the user on the display device.

2. The surgical system of claim 1, wherein the real-time spatial relationship data between the patient's anatomy and the surgical tool includes current positional data of the surgical tool relative to the patient's anatomy.

3. The surgical system of claim 2, wherein the navigation data that the navigation processor is configured to generate is based upon a desired engagement position of the surgical tool relative to the patient's anatomy.

4. The surgical system of claim 3, wherein the navigation data generated by the navigation processor is in the form of a navigation path of the surgical tool relative to the patient's anatomy.

5. The surgical system of claim 4, wherein the navigation processor is configured to display the preferred navigation path on the display device with a label that conveys to the user that it is safe for the surgical tool to be moved along the navigation path.

6. The surgical system of claim 1, wherein the image processor is incorporated into one or more of the cannula assemblies.

7. The surgical system of claim 1, wherein the spatial processor is incorporated into one or more of the cannula assemblies.

8. The surgical system of claim 1, wherein the navigation processor is incorporated into one or more of the cannula assemblies.

9. The surgical system of claim 1, wherein at least one of the image processor, the spatial processor and the navigation processor is incorporated into a control device that is external to the cannula assemblies.

10. The surgical system of claim 9, wherein the control device that is external to the cannula assemblies is configured to transmit and/or receive one or more of wired or wireless data signals from and/or to the cannula assemblies.

11. A cannula assembly for performing a surgical procedure on a patient, comprising:
a cannula tube having a longitudinal axis, a proximal end portion and a distal end portion configured for insertion into a patient,
the cannula tube having a housing coupled to the cannula tube between the proximal and distal ends of the cannula tube so as to be positioned within the patient when the distal end of the cannula tube is inserted into the patient,
the housing movable relative to the cannula tube between a closed position and an open position, the housing including a light source and an image sensor configured to provide a first image data stream of a patient's anatomy and a surgical tool when the housing is in the open position within the patient; and
a processor configured to the process the first data image stream and to generate navigation data based on the image data stream.

12. The cannula assembly of claim 11, wherein the processor is configured to receive a second image data stream from a second imaging device that provides the second image data stream of the patient's anatomy and the surgical tool, the processor configured to process the first and second image data streams into a combined image data stream of the patient's anatomy and the surgical tool.

13. (canceled)

14. The cannula assembly of claim 11, wherein the processor is configured to generate, based upon the combined image data stream of the patient's anatomy and the surgical tool, real-time positional data relative to the patient's anatomy and the surgical tool.

15. The cannula assembly of claim 14, wherein the positional data includes current position data of the surgical tool and the patient's anatomy.

16. The cannula assembly of claim 15, wherein the positional data includes desired engagement position data of the surgical tool and the patient's anatomy.

17. The cannula assembly of claim 16, wherein the navigation data generated by the navigation processor relates to a preferred navigation path of the surgical tool relative to the patient's anatomy from the current position to the desired engagement position.

18. The cannula assembly of claim 17, wherein the processor is configured to display the preferred navigation path on the display device with a label that conveys to the user that it is safe for the surgical tool to be moved along the navigation path.

19. The cannula assembly of claim 11, wherein the processor is incorporated into the cannula assembly.

20. The cannula assembly of claim 11, wherein the processor is external to the cannula assembly.

21. The cannula assembly of claim 11, wherein the processor comprises various controller devices that are both internal and external relative to the cannula assembly.
